(19) United States
(12) Patent Application Publication
(10) Pub. No.: US 2021/0057097 A1
(43) Pub. Date: Feb. 25, 2021

(54) DETECTION OF PRODUCT RESTRICTIONS

(71) Applicant: INTERNATIONAL BUSINESS MACHINES CORPORATION, Armonk, NY (US)

(72) Inventors: Merve Unuvar, Manhattan, NY (US); Henry C. Will, IV, Dover, NJ (US); Suzanne Olivia Livingston, Charlestown, MA (US); Patrick Lowry, Littleton, MA (US)

(21) Appl. No.: 16/546,422

(22) Filed: Aug. 21, 2019

(51) Int. Cl. G16H 50/20; G16H 20/60; G16H 10/20; G06F 3/0482

(52) U.S. Cl. CPC G16H 50/20; G06F 3/0482; G16H 10/20; G16H 20/60

(57) ABSTRACT

A method, computer system, and a computer program product for identifying one or more item restrictions of a user. The present invention may include obtaining a plurality of item data from one or more ingested items associated with the user by utilizing a blockchain structure. The present invention may then include obtaining a plurality of symptom data associated with one or more symptoms of the user. Further, the present invention may also include predicting the one or more item restrictions associated with the user based on the obtained plurality of item data from the one or more ingested items and the obtained plurality of symptom data.

### BACKGROUND

The present invention relates generally to the field of computing and more particularly to diagnostic healthcare.

Increasingly people are discovering that they have a lack of tolerance or even an allergy to certain items. Traditional methods to determine the offending item(s) (e.g., environmental stimuli, food) may be very time consuming with questionable levels of accuracy. For example, in cases involving a possible food restriction, the patient may begin with a very restrictive diet, and, over a period of time, new foods may be slowly introduced to the patient until symptoms return. At that point, a determination may be derived on the particular food restriction of the patient based on which food item was consumed by the patient when the symptom returned.

### SUMMARY

Embodiments of the present invention disclose a method, computer system, and a computer program product for identifying one or more item restrictions of a user. The present invention may include obtaining a plurality of item data from one or more ingested items associated with a user by utilizing a blockchain structure. The present invention may then include obtaining a plurality of symptom data associated with one or more symptoms of the user. Further, the present invention may also include predicting one or more item restrictions associated with the user based on the obtained plurality of item data from the one or more ingested items and the obtained plurality of symptom data.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flowchart illustrating a process for a diagnostic program according to at least one embodiment;

FIG. 3 is an example blockchain architecture configuration according to at least one embodiment;

FIG. 4 is an example graphical user interface (GUI) for symptom data according to at least one embodiment;

FIG. 5 is a block diagram of internal and external components of computers and servers depicted in FIG. 1 according to at least one embodiment;

FIG. 6 is a block diagram of an illustrative cloud computing environment including the computer system depicted in FIG. 1, in accordance with an embodiment of the present disclosure; and

FIG. 7 is a block diagram of functional layers of the illustrative cloud computing environment of FIG. 6, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language, python programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (i.e., LAN) or a wide area network (i.e., WAN), or the connection may be made to an external computer (for example, through the Internet using an Internet Service Provider). In some embodiments, electronic circuitry including, for example, programmable logic circuitry, field-programmable gate arrays (i.e., FPGA), or programmable logic arrays (i.e., PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for identifying item restrictions of a user. As such, the present embodiment has the capacity to improve the technical field of diagnostic healthcare by utilizing a blockchain structure together with analytic methods to both decrease the time utilized to discover an item restriction (i.e., product restriction) and increase the accuracy of the diagnosis made to the user. More specifically, the present invention first obtains data regarding the one or more ingested items that the user is consuming, will consume, or has consumed within a short period of time, from a blockchain structure. The present invention then obtains data associated with one or more symptoms that the user is experiencing. Further, the present invention, utilizing the obtained items data and symptoms data associated with the user, predicts one or more item restrictions (e.g., food or environmental allergies, food or environmental sensitivities, intolerance) associated with the user.

Therefore, it may be advantageous to, among other things, implement a technique to identify item restrictions of a user by utilizing an analytical approach with a blockchain structure.

According to at least one embodiment, the diagnostic program may track one or more ingested items that a user has ingested or is presently ingesting (i.e., ingested items associated with the user) in addition to the digestive or reactive issues that resulted after the user ingested the one or more ingested items. The diagnostic program, in such embodiment, may receive data input from a user of a computing device. Data input may be in the form of a verbal description, a textual description, or an image. The diagnostic program may interpret the data input utilizing speech recognition, natural language processing (i.e., NLP), image recognition, or a machine learning (ML) algorithm designed for the diagnostic program.

According to at least one embodiment, the diagnostic program may obtain data from the tracked ingested items (i.e., item data). Item data may include ingredients, an origin, elements of time (e.g., the time at which such the ingested items were harvested, the length of time the ingested items were in transit, the length of time the ingested items were in storage) and any treatment used on the ingredients (e.g., pesticides used, the process in which ingested items were washed). To obtain item data, the diagnostic program may utilize a blockchain architecture configuration (i.e., blockchain). In such embodiment, the blockchain allows users to obtain item data subsequent to a user (e.g., a manufacturer) inputting such information. The blockchain technology, by design, may provide a trust that the item data to be utilized by the diagnostic program is truthfully stated, not tampered with nor changed. The utilized blockchain structure may execute and utilize permissible frameworks and tools, for example, Hyperledger® (Hyperledger and all Hyperledger—based trademarks and logos are trademarks or registered trademarks of The Linux Foundation non-profit corporation, and/or its affiliates) implementations, such as Hyperledger® Explorer, and Hyperledger® Sawtooth, which are open source projects that collaborate with multiple industry leaders and various software developing resources to leverage blockchain technology for various businesses or industries. Such implementations may allow, in the present invention, manufacturers and processors to privately share item data to the blockchain structure, denying other manufacturers and processors from accessing such private data.

According to at least one embodiment, the diagnostic program may obtain a plurality of symptoms that a user has experienced (i.e., symptom data). Symptom data may include the presence of any nausea, burning sensations, fevers, irregular bowel movements, rashes, cramps, bloating, swelling, wheezing, light-headedness, and a throat closing. Symptom data, in such embodiment, may be obtained upon manual user input of an application of the computing device associated with the user. In further embodiments, symptom data may be obtained automatically through the use of a monitoring unit associated with the user.

According to at least one embodiment, the diagnostic program, subsequent to obtaining item data and the symptoms that the user has experienced, may analyze the ingested items associated with a user. In such embodiment, analysis of the ingested items and the corresponding ingredients may enable the diagnostic program to identify common ingredients between the ingested items or ingredients of such ingested items that may have caused the user to become ill or experience a medical condition. Such analysis may be performed an analytical system, a ML algorithm or a cognitive process.

In reference to at least one embodiment, the diagnostic program may alert a user of the ingested items that may be dangerous to a user (e.g., user has an allergy, sensitivity, intolerance or other form of restriction to the ingested items or corresponding ingredients used in the ingested items) before the user ingests such items. In such embodiment, the diagnostic program may obtain data from the blockchain and further, compare the obtained data associated with the dangerous ingested items, obtained by the blockchain, to any input of a user. Input of a user may be textual data, verbal data, locational data (e.g., the user is located at a restaurant that serves food containing the dangerous ingested items or corresponding ingredients) or image data. For example, if a user scans a barcode at a grocery store of some ingested items that may prove dangerous to the user, the diagnostic program may alert the user to not ingest such ingested items.

In at least one embodiment, the diagnostic program may alert a user based on added item restrictions of the user (e.g., lower caffeine or salt restrictions). In such embodiments, the diagnostic program may analyze such input restrictions, and after obtaining item data, alert the user such restriction level has been reached or is about to be reached. For example, if a doctor recommends a user to limit sodium levels to 1500 milligrams per day, when the user scans the barcode of a box of pizza that reads such pizza has 600 milligrams of sodium, the diagnostic program sends the following to the computing device of the user: “You have already consumed 1000 milligrams of sodium today, so if you order such, you will exceed your 1500 milligrams of sodium per day limit”.

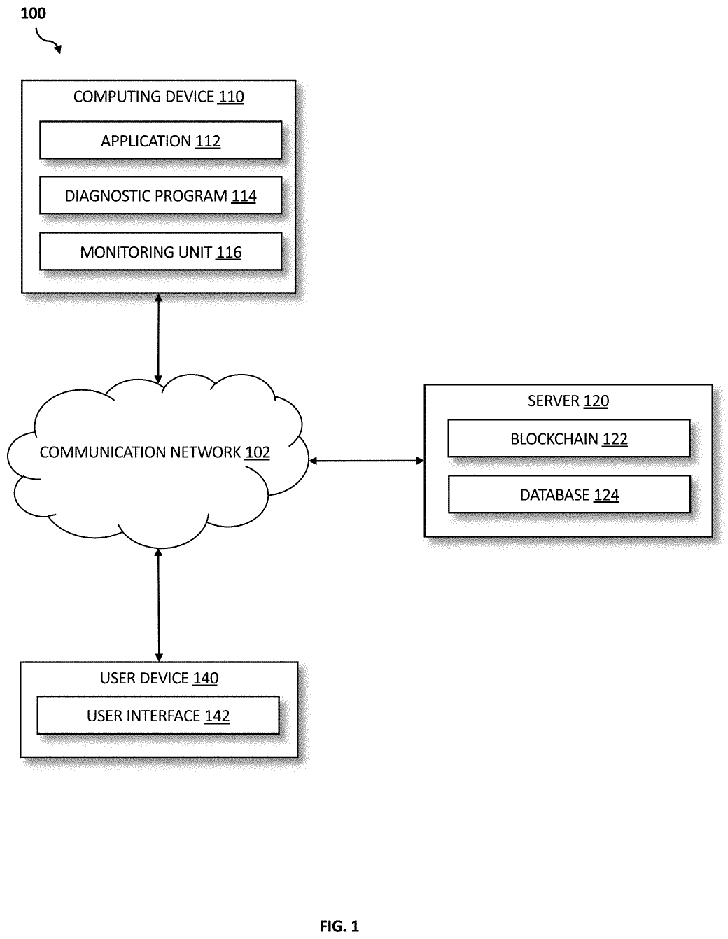

Referring to FIG. 1, an exemplary networked computer environment 100 in accordance with one embodiment is depicted. The networked computer environment 100 may include a computing device 110, a server 120, and a user device 140, interconnected via a communication network 102. While, in the example embodiment, programming and data of the present invention are stored and accessed remotely across several servers via the communication network 102, in other embodiments, programming and data of the present invention may be stored locally on as few as one physical computing device or amongst other computing devices than those depicted.

In the example embodiment, the communication network 102 is a communication channel capable of transferring data between connected devices. The communication network 102 may be the Internet, representing a worldwide collection of networks and gateways to support communications between devices connected to the communication network 102. Moreover, the communication network 102 may include, for example, wired, wireless, or fiber optic connections which may be implemented as an intranet network, a wide area network (WAN), a local area network (LAN), a telecommunication network, a wireless network, a public switched network, a satellite network, or any combination thereof. In general, the communication network 102 may be any combination of connections and protocols that will support communications between the computing device 110, the server 120, and the user device 140. It should be appreciated that FIG. 1 provides only an illustration of one implementation and does not imply any limitations with regard to the environments in which different embodiments may be implemented. Many modifications to the depicted environments may be made based on design and implementation requirements.

In the example embodiment, the computing device 110 includes an application 112, a diagnostic program 114 and a monitoring unit 116 and may be a server, a laptop computer, a netbook computer, a personal computer (PC), a desktop computer, a personal digital assistant (PDA), a rotary phone, a touchtone phone, a smart phone, a mobile phone, a virtual device, a thin client, or any other electronic device or computer system capable of receiving and sending data to and from other computing devices. While, in the example embodiment, the computing device 110 is shown as a single device, in other embodiments, the computing device 110 may be comprised of a cluster or plurality of computing devices, working together or working separately. In various embodiments, the networked computer environment 100 may include one or more of the computing device 110, wherein a user of the computing device 110 may send data to the server 120 or the user device 140.

In the example embodiment, the application 112 is a software program that supports communications between one or more users of the diagnostic program 114 via the user computing device 110, the server 120, and the user device 140, utilizing the communication network 102. In general, the application 112 is a platform in which users can interact with the diagnostic program 114 of the computing device 110 to both provide input and feedback and obtain conclusive results from the diagnostic program 114. The application 112 is also capable of communicating with and transferring data to and from the server 120 and the user device 140 via the communication network 102.

The diagnostic program 114, in the example embodiment, is a software program that supports communications between one or more users of the computing device 110, the server 120, and the user device 140. The diagnostic program 114 is capable of obtaining data regarding a plurality of items associated with a user in addition to item data. Further, the diagnostic program 114 is capable of obtaining symptoms experienced by a user, determining whether the user has a user profile, creating a user profile for the user, predicting item restrictions of the user, providing conclusive results regarding the predicted item restrictions of the user, and finally, receiving and interpreting feedback of a user.

The monitoring unit 116, in the example embodiment, may be comprised of a combination of devices, subsystems, or modules wherein such devices measure events or detect changes in the environment in which the device is being used. Devices, subsystems, or modules may include a plurality of Internet of Things (IoT) sensors, biometric sensors, accelerometers, speedometers, image sensors, or organoleptic sensors. In embodiments, the monitoring unit 116 may communicate with other devices in the networked computer environment 100 to transfer data and command programs to run, in response to obtaining a desired event or a desired change. In the example embodiment, the data collected from the monitoring unit 116 may be stored locally on the computing device 110. In other embodiments, the data collected from the monitoring unit 116 may be stored remotely and accessed via the communication network 102. In the example embodiment, the monitoring unit 116 may be used to detect the physiological state (e.g., functions of the user's body) and physical state (e.g., the user's body itself) of a user. The state of a user may include the monitoring results of a heart rate, a blood pressure reading, a temperature, a galvanic skin response, a stress level, a motion, and an acceleration of a user. The monitoring unit 116 may be comprised of a cluster of plurality of computing devices, working together, or working separately.

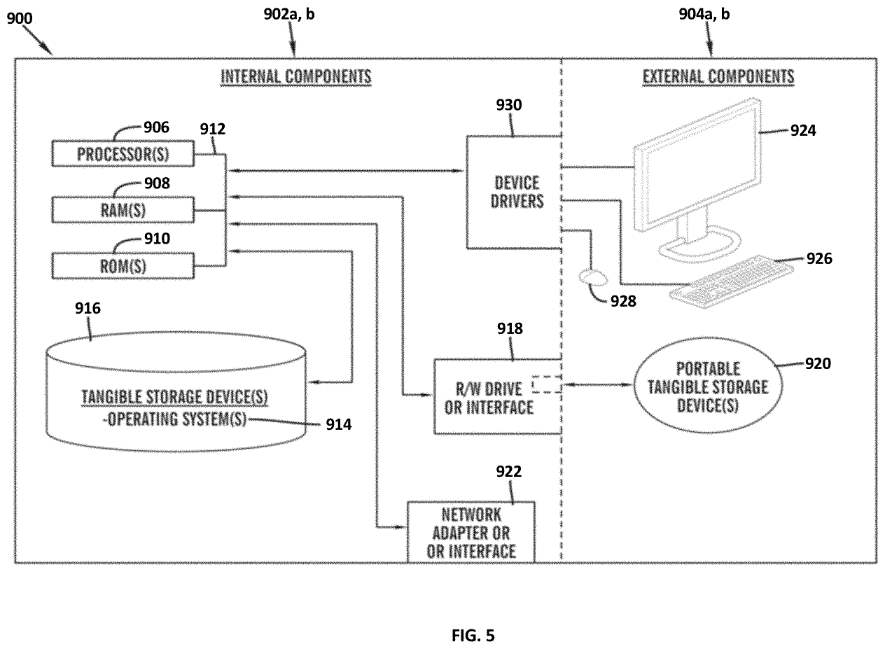

In the example embodiment, the server 120 includes a blockchain 122 and a database 124 and is a device that is capable of communicating with the computing device 110 and the user device 140 via the communication network 102. As will be discussed with reference to FIG. 5, the server 120 may include internal components 902a and external components 904a, respectively. The server 120 may also operate in a cloud computing service model, such as Software as a Service (SaaS), Analytics as a Service (AaaS), Platform as a Service (PaaS), or Infrastructure as a Service (IaaS). The server 120 may also be located in a cloud computing deployment model, such as a private cloud, community cloud, public cloud, or hybrid cloud.

Blockchain technology, in general, may be used for a network system to have consensus, authenticity, immutability and finality of the logged events. The blockchain 122, in the example embodiment, is a collection of files including, but not limited to, hypertext markup language (i.e., HTML) files, cascading style sheets (i.e., CSS) files, extensible markup language (i.e., XML) files, and JavaScript files. In general, the blockchain 122 is used to store information about products or items (e.g., ingredients, origins, treatments, timeline data). In the example embodiment, the blockchain 122 is comprised of a web of a plurality of users capable of accessing a plurality of data. In the example embodiment, the blockchain 122 may be private, restricting access of some parts of the blockchain 122 from a plurality of users (e.g., manufacturer 1 data restricted from manufacturer 2).

The database 124, in the example embodiment, may be comprised of a plurality of HTML files, CSS files, XML files, and JavaScript files. The database 124, in the example embodiment, may include medical records for a plurality of users. In the example embodiment, user profiles (i.e., medical records, prior data) may be associated with a user via a username and password set by a user, an ID number (e.g., the social security number of a user), or a keyword (e.g., the name of the user).

In the example embodiment, the user device 140 includes a user interface 142 and may be a server, a laptop computer, a netbook computer, a personal computer (PC), a desktop computer, a personal digital assistant (PDA), a rotary phone, a touchtone phone, a smart phone, a mobile phone, a virtual device, a thin client, or any other electronic device or computer system capable of receiving and sending data to and from other computing devices. While, in the example embodiment, the user device 140 is shown as a single device, in other embodiments, the user device 140 may be comprised of a cluster or plurality of computing devices, working together or working separately. In various embodiments, the networked computer environment 100 may include one or more of the user device 140, wherein a user of the user device 140 may send data to the computing device 110 or the server 120.

The user interface 142, in the example embodiment, allows the user of the user device 140 to interact with the internal processes of the user device 140 and communicate with other computing devices of the networked computer environment 100. In general, the user interface 142 may be used to obtain, store, and transfer input data from a user (e.g., data regarding a plurality of items from a user).

It should be appreciated that FIG. 1 provides only an illustration of one implementation and does not imply any limitations with regard to the environments in which different embodiments may be implemented. Many modifications to the depicted environments may be made based on design and implementation requirements.

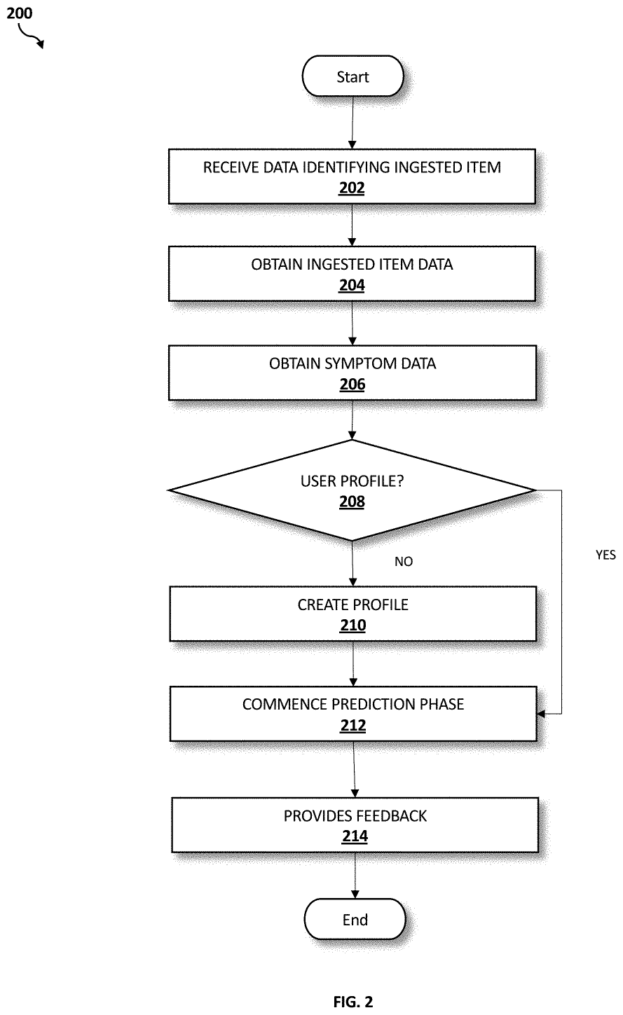

Referring now to FIG. 2, an operational flowchart 200 illustrates the operations of the diagnostic program 114 of the networked computer environment 100, according to at least one embodiment.

At 202, data identifying an ingested item is received. In the example embodiment, the diagnostic program 114 may receive raw data identifying an ingested item (i.e., ingested item) associated with a user of the computing device 110. In the example embodiment, a user may utilize the application 112 to manually input data (i.e., information) regarding an ingested item associated with the user, that may then be transferred to the diagnostic program 114. Manual input of 202 may include verbal data (e.g., “I just ate a slice of pizza”), textual data (e.g., user enters a description or the name of the ingested item in a user's computer or user device and the user's computer or device prompts the user to obtain additional data associated with the received data identifying an ingested item), image data (e.g., a photograph of the item, a photograph of the manufactured item packaging), scanned data (e.g., a barcode of the manufactured item packaging), or physical data (e.g., a piece of item input into an organoleptic sensor).

In at least one embodiment, an ingested item may include any item taken into the body via swallowing or absorbing by the user (e.g., beverages, candy, pharmaceutical drugs, cosmetics, medications, topical creams/ointments), or other contacted substances (e.g., items in which the user comes in contact with).

With verbal and textual data, the diagnostic program 114 may utilize natural language processing (NLP) techniques to determine the context and information collected from the verbal data and/or textual data. More specifically, for NLP, an external engine may utilize an NLP technique (e.g., structure extraction, language identification, tokenization, decompounding, lemmatization/stemming, acronym normalization and tagging, entity extraction, phrase extraction) to process the collected verbal and/or textual data. Then, individual words, phrases, and/or sentences, as well as the relationships between the individual words, phrases and/or sentences, may be extracted from the processed verbal and/or textual data by utilizing various extraction approaches (e.g., top down, bottoms up, statistical). As a result, the diagnostic program 114 interpret the context and meaning for the words, phrases and/or sentences collected by the verbal and/or textual data.

With image data, the diagnostic program 114 may utilize one or more image recognition and image processing tools (e.g., convolutional neural networks (CNNs), pattern recognition) to identify objects (e.g., ingested item(s)) shown in an image. In various image recognition and image processing tools, an image may be broken down in a number of tiles that is individually analyzed, or classified into objects or classes based on key features, to determine the identity of the objects presented in the image.

In embodiments, application 112 may utilize a graphical user interface (i.e., GUI) to allow users to input data. GUIs, in general, allow users to interact with electronic devices through graphical icons and visual indicators, rather than typed command labels or text navigation.

In at least one other embodiment, the diagnostic program 114 may automatically detect the data regarding the ingested item from a social media post associated with the user, or credit card data associated with the user. For example, the user uploads an image of an ingested item on the user's social media, and indicates via textual data or location data that the user is eating or about to eat the ingested item, the diagnostic program 114 may automatically detect such ingested item. In another example, if the user purchases a candy bar at a grocery store and uses a credit card or mobile payment service, then the diagnostic program 114 may automatically detect the purchased ingested item.

In other embodiments, the diagnostic program 114 may automatically detect the data regarding the ingested item. Automatic detection of data regarding the ingested item associated with the user may utilize the monitoring unit 116. For example, a motion sensor associated with the user (e.g., a camera) may detect the user has inserted an object (i.e., an ingested item) into the mouth of the user, or may identify the food on the user's plate, or in the user's hands, while such user is eating. Upon detecting such motion, a camera may take a picture of the ingested item and store the taken image as data associated with the item ingested by the user. As another example, a user may input a food schedule that the user is intends to follow (e.g., a dietary schedule mapping out breakfast, lunch, and/or dinner). The schedule may include a list and/or a description of the ingested items that the user will eat and the time of day the user will eat the ingested items. In such example, the monitoring unit 116 monitors the time of day and upon detecting that the time of day corresponds with the scheduled time of day the user had planned to eat the food, may obtain data on the ingested items the user planned to eat at the scheduled time. In such example, the monitoring unit 116 communicates with the diagnostic program 114 to interpret and analyze the obtained times of day.

In the present embodiment, where the diagnostic program 114 automatically detects the ingested items associated with the user, the diagnostic program 114 may prompt the user to confirm that the identity of the automatically detected ingested items associated with a user is accurate. The user may confirm the identity of the detected ingested item utilizing the user interface 142.

In one embodiment, the diagnostic program 114 may continue to obtain data associated with the ingested item after a defined time (e.g., five minutes), regardless of whether the user has confirmed the identity of the ingested item. In another embodiment, the diagnostic program 114 may not continue to obtain data on the ingested item until the user has confirmed that the automatically detected ingested item associated with a user is accurate.

In at least one embodiment, if the diagnostic program 114 determines the user has disproved of one or more automatically detected ingested items (e.g., the user decided not to follow the input schedule), the automatically one or more detected ingested items associated with a user may be removed from any data obtained on the ingested items associated with the user.

As an illustrative example, User A prepares and consumes a personal pizza manufactured by Manufacturer A. The User A uses a smart phone to scan the barcode on the packaging of the personal pizza.

At 204, item data is obtained. In the example embodiment, the diagnostic program 114 first collects the data associated with the ingested item associated with the user (i.e., ingested item data). Such data, in the example embodiment, may be transferred locally, without utilizing the communication network 102. In other embodiments, where the diagnostic program 114 is located on a remote computing device separate and distinct from the computing device 110, the obtained data may be transferred via the communication network 102. The diagnostic program 114 may analyze the obtained data regarding the ingested items associated with the user. In the example embodiment, the diagnostic program 114 may utilize natural language processing for speech recognition and textual understandings, in addition to, image recognition, or image processing.

In the example embodiment, the diagnostic program 114 may parse through the obtained ingested item data associated with the user for data and obtains the item data associated with the obtained ingested item data from the blockchain 122. Item data may include the ingredients, an origin, elements of time (e.g., the time at which such the ingested item was harvested, the length of time the ingested item was in transit, the length of time the ingested item was in storage) and the treatment of the ingested item and corresponding ingredients (e.g., pesticides used, the process in which ingested items were washed). The blockchain 122 may include a web of multiple users who are capable of accessing data on a plethora of ingested items and corresponding ingredients. Prior, during, or subsequent to the activation of the diagnostic program 114, permissible users of the blockchain 122 may input data into the blockchain 122 (e.g., manufacturers may input item data about the processed ingested items manufactured into the web).

In embodiments, where the item data associated with the received data identifying the ingested items associated with the user may be excluded from the blockchain structure (e.g., the Manufacturer E of the input food F has no stored blockchain input data), the diagnostic program 114 may proceed to obtain symptom data at 206.

Furthering the previous example, the scanned barcode has identified the ingested item the user is about to ingest to be a personal pizza of Manufacturer A. The diagnostic program 114 parses through the blockchain 122 to find item data associated with the personal pizza of Manufacturer A. Manufacturer A upon distribution of the pizzas to stores, input the following ingredients, origin, and elements of time of the personal pizza to the blockchain 122:

TABLE 1

| Identified | Personal Cheese and Mushroom Pizza | | | |
|---|---|---|---|---|
| Food | | | | |
| Ingredients | feta | tomato | mushrooms | pizza dough (wheat flour, |
| | cheese | sauce | | yeast, sugar, salt) |
| Origin | farm A | farm B | farm B | manufacturer B |

Harvest time = 4 days prior
Transit time = 2 days
Storage time = 2 days in freezer, 1 day out of freezer
Processing = all ingredients washed with purified water

Table 1 represents the item data obtained from the personal pizza of Manufacturer A.

At 206, the symptom data is obtained. In the example embodiment, the diagnostic program 114 may automatically obtain symptom data from a user of the computing device 110 utilizing a monitoring unit 116. The monitoring unit 116, in some embodiments, may include multiple IoT sensors, biometric sensors, accelerometers, speedometers, image sensors, or organoleptic sensors. In general, the monitoring unit 116 may detect the physiological and physical state of a user. Symptom data obtained may include heart rate, blood pressure, temperature, galvanic skin response, stress level, motion, the presence of a rash, breathing patterns, and acceleration of a user. Symptom data may also include the length of time the symptom occurred, how soon the reaction came about after ingesting the ingested items by the user, and the order or sequence in which the symptoms were experienced.

In the example embodiment, the monitoring unit 116 may communicate with the diagnostic program 114 locally. In other embodiments, where the diagnostic program 114 may be stored on a remote device, the monitoring unit 116 may communicate with the diagnostic program 114 utilizing the communication capabilities of the communication network 102.

In other embodiments, symptom data of a user may be collected via manual input. Manual input may include verbal data (e.g., a user says “My stomach hurts”), textual data, selection data (e.g., check list of the symptoms the user is experiencing), or image data (e.g., the user drawing on a representative body where the user is experiencing such symptoms). In such embodiments, the diagnostic program 114 may utilize natural language processing (NLP), voice recognition, image recognition, or any other ML algorithm designed for the diagnostic program 114 to analyze and interpret the obtained symptom data.

In the example embodiment, the symptom data obtained by the diagnostic program 114 may be stored remotely within the blockchain 122 or the database 124 associated with a user's medical records. In other embodiments, the symptom data may be stored locally on the computing device 110.

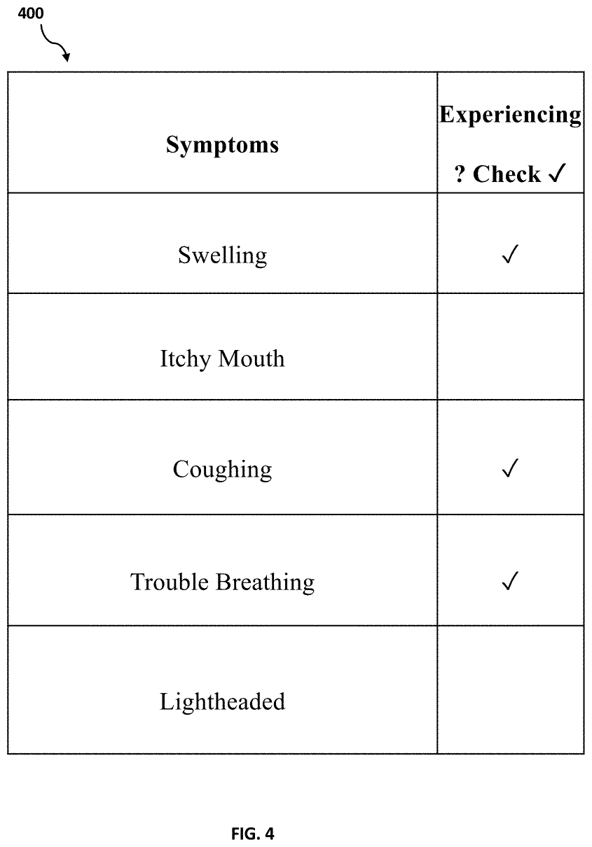

In furthering the previous example, the diagnostic program 114 prompts the User A to check off the symptoms User A may be experiencing on the smart phone diagnosis application. The symptom data provided by User A and collected by the diagnostic program 114 is shown in FIG. 4.

At 208, the diagnostic program 114 determines whether the user of the diagnostic program 114 has a user profile. A user profile may include data associated with the user's determined item restrictions, dietary restrictions (e.g., reduced salt intake) based on a medical recommendation, and obtained item data associated with a previously ingested item with associated symptoms that may not have resulted in a clear determination of the cause (e.g., rice and beans, nauseous) (i.e., past ingested item data). Additionally, a user profile may include the name, age, race, height, weight, and family history of a user of the computing device 110.

In the example embodiment, user profiles may be stored on the blockchain 122. In such embodiments, the diagnostic program 114 may obtain data from the blockchain 122 utilizing the communication network 102. In such embodiments, the diagnostic program 114 may include an opt-in/opt-out feature in which the user may select to opt-in or opt-out from the collection of user profile data associated with the specific user. The opt-in/opt-out feature may be changed at any time, and if the user opts-in to the diagnostic program 114, then the user may be notified when data collection is activated, and the data collection has commenced. In some embodiments, the user may limit when data is collected, how the data is collected, what type of data is collected (e.g., “Other users cannot access my weight”), and the purposes for which the data collected is utilized. The user may also limit how and where the data is stored. In such embodiments, wherein the user profile is stored on the blockchain, the user profile data may be used, with permission from the user, to improve correlations of the diagnostic program 114 for different users.

In other embodiments, user profiles may be stored on the database 124. In such embodiments, the diagnostic program 114 may obtain data from the database 124 via the communication network 102.

In the example embodiment, the diagnostic program 114 may obtain one known attribute of the user of the computing device 110 and may further search the database containing the plurality of user profiles (e.g., the blockchain 122 or the database 124) to find the user profile associated with the obtained known attribute of the user. Attributes may include a user's name, identification (ID) number, username and password, or keyword that distinguishes such user from any other user of the diagnostic program 114.

In furthering the previous example, the diagnostic program 114 utilizes the contact information of the user of the computing device 110 to determine that User A has a social security number of 123456789. The diagnostic program 114 then searches the user profiles associated with the blockchain 122 to find the user profile that contains the social security number 123456789.

If the diagnostic program 114 determines the user of the diagnostic program 114 does not have a user profile at 208 (i.e., decision 208 “NO” branch), the diagnostic program 114 prompts a user of the computing device 110 to create a profile at 210. A user profile may allow the diagnostic program 114 to link the data collected from the activation of the diagnostic program 114 to the user of the computing device 110 during such activation. User profiles may be linked with data of a user via an attribute, which may include, a username and password combination set by a user, an ID number (e.g., the social security number of a user), or a keyword (e.g., the name of the user).

In the example embodiment, the diagnostic program 114 may automatically obtain personal information associated with the computing device 110 of the user (e.g., contact information of the user from a smart phone) to create and link a new user profile to the user of the computing device 110. In other embodiments, the diagnostic program 114 may obtain personal information via manual input of a user of the computing device 110. In such embodiment, the diagnostic program 114 may allow the user to interact with the application 112 of the computing device 110 for textual manual input. The diagnostic program 114 may also allow personal interaction of the monitoring unit 116 of the computing device 110 for obtaining input (e.g., verbal input). The diagnostic program 114 may then analyze such data using methods as in speech recognition, image recognition, and natural language processing. A newly created user profile may be stored in the database wherein the plurality of user profiles may be stored (e.g., the blockchain 122 or the database 124).

In some embodiments, the user of the computing device 110 may choose to opt out of creating a user profile at this time. As such, the diagnostic program 114 may proceed to the prediction phase at 212.

If, however, the diagnostic program 114 determines the user has a user profile at 208 (i.e., decision 208 “YES” branch), the diagnostic program 114 commences the prediction phase at 212. In general, the prediction phase may include the prediction of data that may correlate the obtained ingested item data associated with the user, the obtained item data from the blockchain 122, and the obtained symptom data in order to predict item restriction data. In the example embodiment, the diagnostic program 114 utilizes a ML algorithm to correlate and analyze the ingested item data, the item data, the symptom data, and, when applicable, data stored in the user profile, to further determine which of the one or more ingested items associated with the user is causing negative (e.g., troubling) symptoms. In such embodiment, the diagnostic program 114 may utilize data obtained from the user of the computing device 110. In other embodiments, the diagnostic program 114 may obtain user profile data of anonymous users in addition to the data obtained from the user of the computing device 110. For example, if User A is gluten free and User B is also gluten free, the data obtained from a new ingested item input by User B may apply to User A. In some embodiments, a user of the computing device 110 may interact with the application 112 to select preferences for correlation of data (e.g., a User A selecting that such user prefers to have data from similar users influence the prediction of item restriction). ML algorithms (e.g., multinomial regression, neural networks, multi-dimensional clustering, dimensionality reduction, and model selection) utilized, in some embodiments, may be trained and designed with unsupervised or supervised previous data from other user profiles.

Predictions (i.e., potential causes) may be generated in the form of confidence levels (e.g., “With 75% confidence, the input ingested item may cause headaches”, “With 75% confidence, you are allergic to gluten.”), thresholds (e.g., “The User may experience symptoms after 50 g of the Food A”), categorical values (e.g., “The User may be lactose intolerant”), symptomatic data (e.g., “The input ingested item is likely to cause headaches, bloating, and a stomachache”), alternative data (e.g., “The input ingested item may cause headaches. A similar ingested item may be Food B, which has proven previously not to cause headaches”), or future recommendations (e.g., “It would be best to avoid foods manufactured by Manufacturer A”).

In furthering the previous example, the diagnostic program 114 identifies the user profile associated with the social security number 123456789. The diagnostic program 114 obtains the data from the user profile of the User A. The diagnostic program 114 then utilizes a previously trained neural network to predict for the User A. After training, the neural network assigns weights of importance to each category of obtained data as seen below. The diagnostic program 114 then inputs the following data:

foods associated with the user (0.10)=

personal cheese and mushroom pizza

item data (0.40)=
feta cheese—farm A, 4 days prior, 2 days, 2 days in freezer, 1 day out of freezer, purified water
tomato sauce—farm B, 4 days prior, 2 days, 2 days in freezer, 1 day out of freezer, purified water
mushrooms—farm B, 4 days prior, 2 days, 2 days in freezer, 1 day out of freezer, purified water
pizza dough—manufacturer B, 4 days prior, 2 days, 2 days in freezer, 1 day out of freezer, purified water
   symptoms (0.20)=

bloating

nausea

itchy rash

headache

user profile data (0.30)=

name—User A

social security number—123456789
previously ingested item that has caused symptoms without clear determination of cause—mozzarella cheese

The above data in addition to the weights associated with each category (e.g., ingested items associated with a user—0.10), is utilized by the neural network of the diagnostic program 114. The diagnostic program 114 further predicts, “With an 85% confidence level, the user is lactose intolerant”.

At 214, feedback data is provided. In the example embodiment, where the diagnostic program 114 was able to determine a user profile, feedback data may be stored and linked to the user profile of the user of the computing device 110. Feedback data may include the ingested item data associated with the user, item data from the blockchain 122 that corresponds with the ingested item data, symptom data, and the predictions received from the diagnostic program 114.

In the embodiments, where the diagnostic program 114 was unable to determine a user profile, feedback data may be stored and linked to a created user profile at 210 of the user of the computing device 110. In some embodiments, where the user chose to opt out of creating a user profile, the diagnostic program 114 may store the data anonymously. Such anonymous data may be stored remotely in the blockchain 122 of the server 120 or the database 124 of the server 120. Anonymous data, with permission of the user of the computing device 110, may be stored and with later activation of the diagnostic program 114 by other users, may be obtained, utilized, and correlated to increase the accuracy of the predictions.

In furthering the previous example, the diagnostic program 114 may store in the identified user profile of User A that the user is lactose intolerant. Such information may be updated in the blockchain 122. With the User A's permission, the doctor observes such predictions and updating user's records as applicable.

In another embodiment, where the ingested item data that corresponds with the item data associated with the user cannot be found in blockchain 122, the diagnostic program 114, when providing feedback at 214, may flag the prediction made by the diagnostic program 114. The flag may alert the user, if such user were to obtain the ingested item data again, that such prediction was made without known item data. In such embodiment, the diagnostic program 114, upon receiving input to blockchain 122 from the manufacturer of the items associated with the user that previously did not have known item data, may identify the flagged prediction and may further re-correlate, re-analyze and re-predict item restrictions utilizing the new input of blockchain 122.

For example, a User D may have eaten a Cheese F of Manufacturer E at a first period of time. At such first time period, Manufacturer E had not input any product or item information to the blockchain structure. Therefore, the prediction regarding the effect of Cheese F on the User M was made without knowing the item data. Because of insufficient data from Manufacturer E, the prediction had been flagged within the profile of User D. At a second period of time, the Manufacturer E has input the item data associated with Cheese F to the blockchain structure. At the second period of time, the diagnostic program 114 may obtain the flagged prediction associated with the Manufacturer E. Utilizing the combination of the plurality of data obtained at a first period of time and the plurality of data obtained at a second period of time, the diagnostic program 114 predicts again the effect of Cheese F on the User M.

In the present embodiment, the diagnostic program 114 may include external and internal allergic conditions caused by external stimuli (e.g., scents, environmental allergens, trees, mold, pollen) that may be tracked in a similar manner, such as textiles, soap or detergents.

In the present embodiment, the predictions generated, and various types of data collected by diagnostic program 114 may be utilized by product manufacturers, suppliers, distributors, shippers, and other key roles in the supply chain to assess whether there exists an issue, or potential issue, with shipping processes, handling or ingredients. Therefore, whether such processes or ingredients may be modified or eliminated from the supply or product chain in a particular item. For example, if a large number of users are feeling ill after eating a salad in multiple different states across the United States, the data collected by the diagnostic program 114 may be able to determine that the commonality between these users is that each salad included arugula, and although the arugula came from different farms, the arugula took eight days, as opposed to six days, to reach the various grocery stores. Therefore, the distributors may evaluate their shipping processes to reduce the transit time of the arugula and potentially avoid users becoming ill due to the extended transit times for arugula.

The functionality of a computer may be improved by the diagnostic program 114 because the diagnostic program 114 addressing an alternate means of identifying item restrictions of a user by combining analytics and blockchain technology. The analytics may be utilized to evaluate the symptoms exhibited by a person based on the ingested items and corresponding ingredients of the ingested items taken in or absorbed by the person to determine the ingested item that may have contributed to, or caused, the symptoms (i.e., potential cause). Blockchain may be utilized to provide a trust that the ingredients in the ingested items have been disclosed honestly. The blockchain may also provide a means for tracking an ingested item or corresponding ingredient throughout the product chain from growth, harvest, transport and final preparation thereby directly determining the source or cause of the symptom based on not only the type of ingested item or ingredient ingested by the person, but the treatment of such ingested item or ingredient prior to ingestion.

Additionally, the diagnostic program 114 may identify these item restrictions is a more time efficiency (e.g., less time consuming) matter with an increase in accuracy (e.g., less questionable results). Furthermore, the diagnostic program 114 may utilize Internet of Things (IoT) devices and biometric sensors to detect the ingestion of the ingested item, and whether a location includes such ingested item that was previously attributed to a negative reaction or symptom exhibited by the user (e.g., a person who may exhibit symptoms with the mere smell or touch of seafood or peanuts).

The diagnostic program 114 may further improve computer functionality by a person's item restriction to an unknown ingested item based on the relationship with the unknown ingested item and another ingested item in which the user exhibited a symptom or reaction to, even if the user has never ingested the unknown ingested item or ingredient.

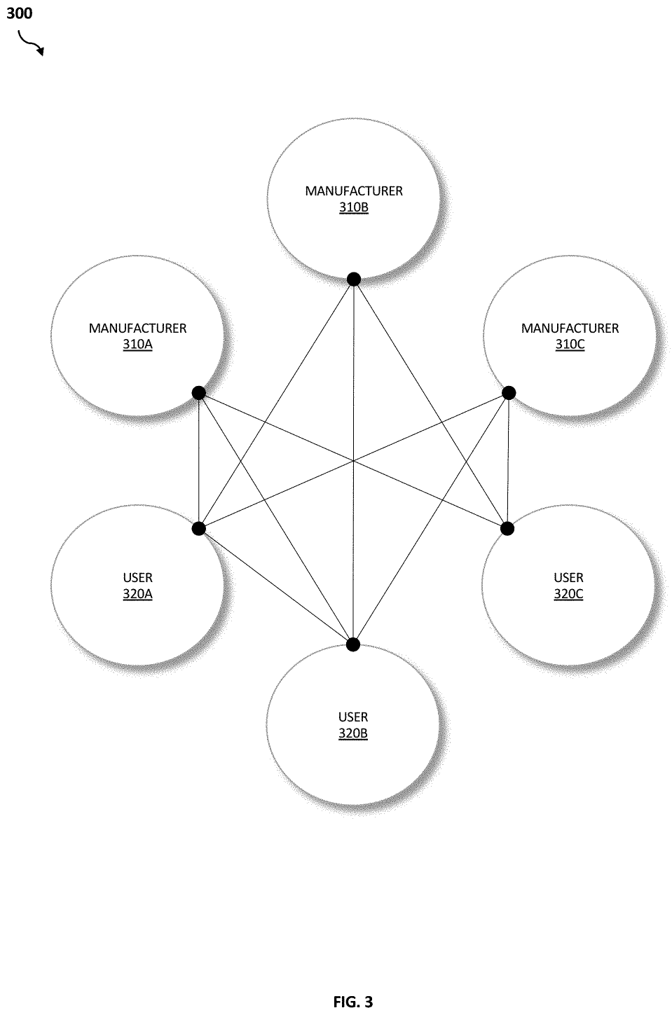

FIG. 3 is an example blockchain architecture configuration depicted in FIG. 1 according to at least one embodiment. It should be appreciated that FIG. 3 provides only an illustration of one implementation and does not imply any limitations with regard to the environments in which different embodiments may be made based on design and implementation requirements.

Blockchain technology may include a shared log of events that are kept in blocks of data that may be passed to the next transaction in a linear order. A block may contain the data of one or more records or transactions. A block may hold or store the data before the block has been completed, prior to consensus and prior to being transferred to the next block in a blockchain, for example, using a transaction pool, a memory pool or a candidate block. A block may also be a block of one or more records or transactions attached to a blockchain, for example, a transaction that has been permanently appended and recorded to the files in a blockchain.

With blockchain technology, for example, when a transaction is written into a ledger, there may be a mechanism to make sure all the records are synchronized and if the network system detects an error, the error may be immediately corrected. Blockchain systems are immutable since, for example, a hash value on each block may be created and any change or amendment in data will show a different hash value. Each block may contain the hash of the previous block and the current transactions before the next hash may be created. Once consensus is reached, the data may be verified. Each block may have a hash value of the previous block in the chain and there may be no way to modify the data in the previous block. If the data is modified, the modified data may be detected by a re-calculated hash value. A different hash value may show that the network system has been compromised.

Manufacturer 310A, 310B, 310C are representative of a user of the blockchain 122, where such user may input information to the blockchain regarding the ingested item, and corresponding ingredients, each manufacturer may produce. In the example embodiment, manufacturer 310A, 310B, 310C does not have permissible access to any of the data input by any of the manufacturer 310A, 310B, 310C unique from itself or user 320A, 320B, 320C. For example, manufacturer 310A, 310B, 310C may be a manufacturer representative who has permission to only input item data for users 320A, 320B, 320C to access.

User 320A is representative of a user of the blockchain 122, where such user may input information to the blockchain 122 in addition to accessing information from manufacturers 310A, 310B, 310C. Additionally, user 320A may access information from user 320B. For example, User 320A may be a physician who has permission to access both the user 320B, 320C and data (e.g., the ingested item data associated with the user) in addition to the item data received from the manufacturer.

User 320B, 320C are representative of a user of the blockchain 122, where such user may input information to the blockchain 122 regarding one's own data regarding the products associated with the user, in addition to one's own medical records or personal information. Additionally, a user may access information from manufacturers 310A, 310B and 310C (e.g., item data). For example, User 320B, 320C may be a patient who has permission to access data associated with the manufacturer 310A, 310B, 310C for item data.

FIG. 4 is an example graphical user interface (GUI) 400 for symptom data input by a user of the diagnostic program 114. Such GUI may be on the screen of the smart phone, and the user may be prompted with a list of symptoms. The GUI may allow a user, upon interaction, to input, or check off, which symptoms the user may be experiencing, thereby enabling the diagnostic program 114 to collect symptom data to determine the item restriction of the user. In this example, User A is presented with a list of symptoms, namely swelling, itchy mouth, coughing, trouble breathing and lightheaded. User A placing a finger next to each of the symptoms that User A is experiencing, namely swelling, coughing and trouble breathing. Since the smart phone used by User A is a touch screen in which placing a finger in the “Experiencing?” column next to a symptom places a checkmark in that column thereby indicating that User A is experiencing that symptom.

In at least one embodiment, there may be “More Symptoms” button at the bottom of the screen in which a user may click for the diagnostic program 114 to list more symptoms for the user to review and select if applicable. In some embodiments, at the end of the list of symptoms, the diagnostic program 114 may include a “Submit” button to indicate that the user is finished selecting the present symptoms (i.e., symptoms that the user is presently experiencing). In at least one other embodiment, the diagnostic program 114 may include a time limit for the user indicate the present symptoms (e.g., a default time limit is 60 seconds). Once the time limit has lapsed, the user may be prompted (e.g., via a dialog box) to indicate whether the user is finished selecting symptoms, or would prefer more time. In another embodiment, after the time limit has expired, the diagnostic program 114 automatically closes the GUI and ends the collection of symptom data from the user.

It may be appreciated that FIGS. 2, 3 and 4 provide only an illustration of one embodiment and does not imply any limitations with regard to how different embodiments may be implemented. Many modifications to the depicted embodiment(s) may be made based on design and implementation requirements.

FIG. 5 is a block diagram 900 of internal and external components of computers depicted in FIG. 1 in accordance with an illustrative embodiment of the present invention. It should be appreciated that FIG. 5 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The communication network 102, the computing device 110, the server 120, and the user device 140 may include respective sets of internal components 902a, b and external components 904a, b illustrated in FIG. 5. Each of the sets of internal components 902a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the diagnostic program 114 on the computing device and the application 112 on the computing device, may be stored on one or more computer-readable tangible storage devices 916 for execution by one or more processors 906 via one or more RAMs 908 (which typically include cache memory). In the embodiment illustrated in FIG. 3, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as diagnostic program 114 of the computing device 110 or application 112 of the computing device 110, can be stored on one or more of the respective portable computer-readable tangible storage devices 920, read via the respective R/W drive or interface 918 and loaded into the respective hard drive 916.

Each set of internal components 902a, b may also include network adapters (or switch port cards) or interfaces 922 such as a TCP/IP adapter cards, wireless wi-fi interface cards, or 3G or 4G wireless interface cards or other wired or wireless communication links. The diagnostic program 114 of the computing device 110, in addition to the application 112 of the computing device 110 can be downloaded from an external computer (e.g., server) via a network (for example, the Internet, a local area network or other, wide area network) and respective network adapters or interfaces 922. From the network adapters (or switch port adaptors) or interfaces 922, the diagnostic program 114 of the computing device 110 and the application 112 of the computing device 110 are loaded into the respective hard drive 916. The network may comprise copper wires, optical fibers, wireless transmission, routers, firewalls, switches, gateway computers and/or edge servers.

Each of the sets of external components 904a, b can include a computer display monitor 924, a keyboard 926, and a computer mouse 928. External components 904a, b can also include touch screens, virtual keyboards, touch pads, pointing devices, and other human interface devices. Each of the sets of internal components 902a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Analytics as a Service (AaaS): the capability provided to the consumer is to use web-based or cloud-based networks (i.e., infrastructure) to access an analytics platform. Analytics platforms may include access to analytics software resources or may include access to relevant databases, corpora, servers, operating systems or storage. The consumer does not manage or control the underlying web-based or cloud-based infrastructure including databases, corpora, servers, operating systems or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

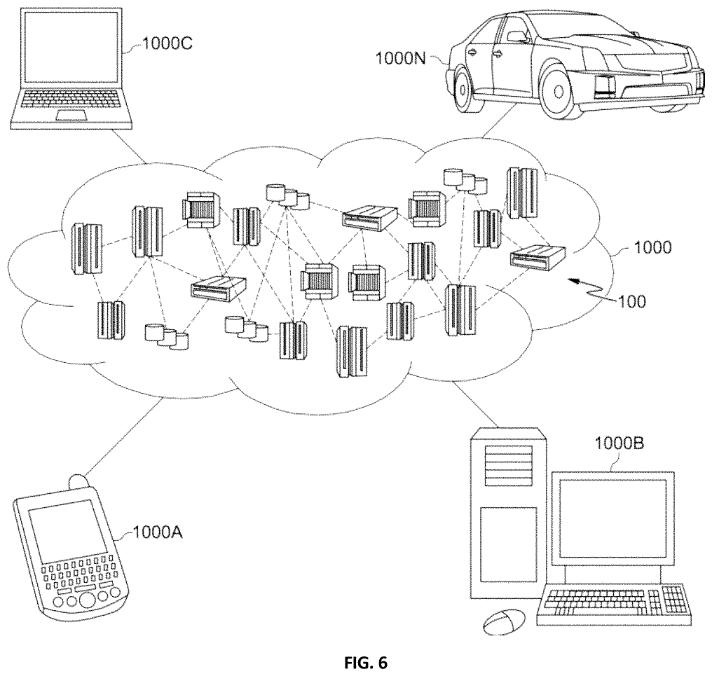

Referring now to FIG. 6, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 6 are intended to be illustrative only and that computing nodes 100 and cloud computing environment 1000 can communicate with any type of computerized device over any type of network and/or network addressable connection (e.g., using a web browser).

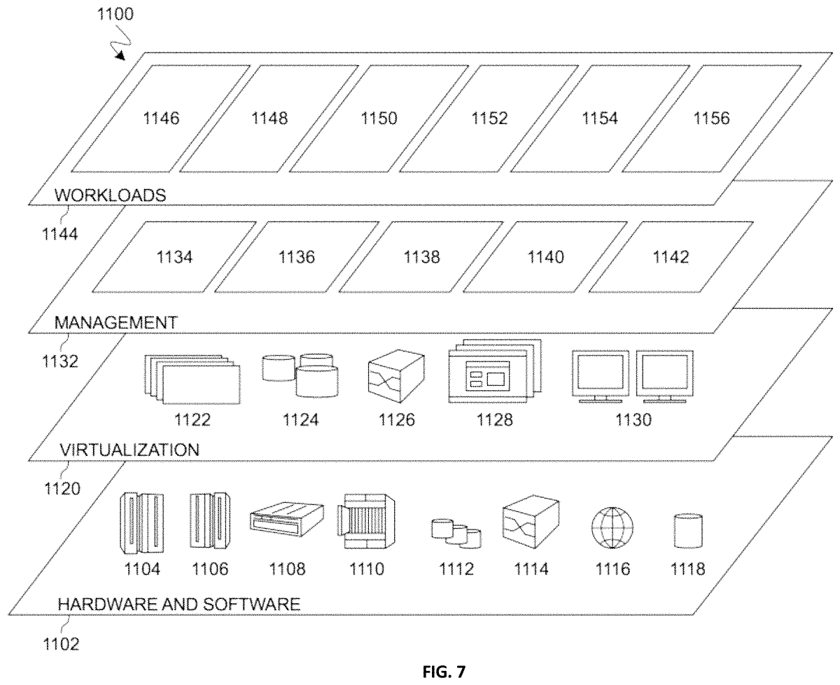

Referring now to FIG. 7, a set of functional abstraction layers 1100 provided by cloud computing environment 1000 is shown. It should be understood in advance that the components, layers, and functions shown in FIG. 7 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture-based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and diagnostic program 1156.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
obtaining a plurality of item data from one or more ingested items associated with a user by utilizing a blockchain structure;
obtaining a plurality of symptom data associated with one or more symptoms of the user; and
predicting one or more item restrictions associated with the user based on the obtained plurality of item data from the one or more ingested items and the obtained plurality of symptom data.

2. The method of claim 1, wherein obtaining the plurality of item data from the one or more ingested items associated with the user by utilizing the blockchain structure, further comprises:
verifying, by a plurality of users, the obtained plurality of item data associated with the one or more ingested items associated with a trust.

3. The method of claim 1, wherein obtaining the plurality of symptom data associated with the one or more symptoms of the user, further comprises:
in response to determining the user does not have a user profile, creating the user profile associated with the user; and
obtaining a plurality of past ingested item data from the created user profile associated with the one or more item restrictions associated with the user.

4. The method of claim 3, further comprising:
predicting the one or more item restrictions associated with the user based on the obtained plurality of past ingested item data from the created user profile and the obtained item data from the blockchain.

5. The method of claim 4, wherein predicting the one or more item restrictions associated with the user with the user based on the obtained plurality of past ingested item data from the created user profile and the obtained item data from the blockchain, further comprises:
analyzing the obtained plurality of symptom data, the obtained plurality of past ingested item data and the plurality of item data associated with the one or more ingested items; and
correlating the analyzed plurality of symptoms data with the analyzed plurality of past ingested item data and the analyzed plurality of item data.

6. The method of claim 5, wherein correlating the analyzed plurality of symptoms data with the analyzed plurality of past ingested item data and the analyzed plurality of item data, further comprises:
analyzing at least one relationship with the correlated plurality of symptoms data, the correlated plurality of past ingested item data and the correlated plurality of item data; and
determining at least one potential cause associated with the one or more predicted item restrictions based on the analyzed relationship.

7. The method of claim 1, further comprises:
receiving one or more ingested items by the user,
wherein the received one or more ingested items include a plurality of ingredients associated with the received one or more ingested items; and
confirming one or more identity associated with the received one or more ingested items.

8. A computer system for identifying one or more item restrictions of a user, comprising:
one or more processors, one or more computer-readable memories, one or more computer-readable tangible storage media, and program instructions stored on at least one of the one or more computer-readable tangible storage media for execution by at least one of the one or more processors via at least one of the one or more memories, wherein the computer system is capable of performing a method comprising:
obtaining a plurality of item data from one or more ingested items associated with the user by utilizing a blockchain structure;
obtaining a plurality of symptom data associated with one or more symptoms of the user; and
predicting the one or more item restrictions associated with the user based on the obtained plurality of item data from the one or more ingested items and the obtained plurality of symptom data.

9. The computer system of claim 8, wherein obtaining the plurality of item data from the one or more ingested items associated with the user by utilizing the blockchain structure, further comprises:
verifying, by a plurality of users, the obtained plurality of item data associated with the one or more ingested items associated with a trust.

10. The computer system of claim 8, wherein obtaining the plurality of symptom data associated with the one or more symptoms of the user, further comprises:
in response to determining the user does not have a user profile, creating the user profile associated with the user; and
obtaining the plurality of past ingested item data from the created user profile associated with the one or more item restrictions associated with the user.

11. The computer system of claim 10, further comprising:
predicting the one or more item restrictions associated with the user based on an obtained plurality of past ingested item data from the created user profile and the obtained item data from the blockchain.

12. The computer system of claim 11, wherein predicting the one or more item restrictions associated with the user with the user based on the obtained plurality of past ingested item data from the created user profile and the obtained item data from the blockchain, further comprises:
analyzing the obtained plurality of symptom data, the obtained plurality of past ingested item data and the plurality of item data associated with the one or more ingested items; and
correlating the analyzed plurality of symptoms data with the analyzed plurality of past ingested item data and the analyzed plurality of item data.

13. The computer system of claim 12, wherein correlating the analyzed plurality of symptoms data with the analyzed plurality of past ingested item data and the analyzed plurality of item data, further comprises:
analyzing at least one relationship with the correlated plurality of symptoms data, the correlated plurality of past ingested item data and the correlated plurality of item data; and
determining at least one potential cause associated with the one or more predicted item restrictions based on the analyzed relationship.

14. The computer system of claim 8, further comprises:
receiving one or more ingested items by the user,
wherein the received one or more ingested items include a plurality of ingredients associated with the received one or more ingested items; and
confirming one or more identity associated with the received one or more ingested items.

15. A computer program product for identifying one or more item restrictions of a user, comprising:
one or more computer-readable storage tangible media and program instructions stored on at least one of the one or more computer-readable tangible storage media, the program instructions executable by a processor to cause the processor to perform a method comprising:
obtaining a plurality of ingested item data from one or more ingested items associated with the user by utilizing a blockchain structure;
obtaining a plurality of symptom data associated with one or more symptoms of the user; and
predicting the one or more item restrictions associated with the user based on the obtained plurality of item data from the one or more ingested items and the obtained plurality of symptom data.

16. The computer program product of claim 15, wherein obtaining the plurality of item data from the one or more ingested items associated with the user by utilizing the blockchain structure, further comprises:
verifying, by a plurality of users, the obtained plurality of item data associated with the one or more ingested items associated with a trust.

17. The computer program product of claim 15, wherein obtaining the plurality of symptom data associated with the one or more symptoms of the user, further comprises:
in response to determining the user does not have a user profile, creating the user profile associated with the user; and
obtaining a plurality of past ingested item data from the created user profile associated with the one or more item restrictions associated with the user.

18. The computer program product of claim 17, further comprising:
predicting the one or more item restrictions associated with the user based on the obtained plurality of past ingested item data from the created user profile and the obtained item data from the blockchain.

19. The computer program product of claim 18, wherein predicting the one or more item restrictions associated with the user with the user based on the obtained plurality of past ingested item data from the created user profile and the obtained item data from the blockchain, further comprises:
analyzing the obtained plurality of symptom data, the obtained plurality of past ingested item data and the plurality of item data associated with the one or more ingested items; and
correlating the analyzed plurality of symptoms data with the analyzed plurality of past ingested item data and the analyzed plurality of item data.

20. The computer program product of claim 19, wherein correlating the analyzed plurality of symptoms data with the analyzed plurality of past ingested item data and the analyzed plurality of item data, further comprises:
analyzing at least one relationship with the correlated plurality of symptoms data, the correlated plurality of past ingested item data and the correlated plurality of item data; and
determining at least one potential cause associated with the one or more predicted item restrictions based on the analyzed relationship.
